(19) United States
(12) Patent Application Publication
(10) Pub. No.: US 2002/0122830 A1
(43) Pub. Date: Sep. 5, 2002

(54) Method for the treatment of a soil containing soilborne pathogens

(76) Inventors: Mario Tenuta, London (CA); George Lazarovits, London (CA)

Correspondence Address: Eric Fincham, 316 Knowlton Road, Lac Brome, QC, J0E 1V0, CA

(21) Appl. No.: 10/017,328

(22) Filed: Dec. 14, 2001

Related U.S. Application Data
(63) Continuation-in-part of application No. 09/624,098, filed on Jul. 24, 2000.

(51) Int. Cl. A61K 35/24; A61K 35/36; A61K 35/34; C05B 17/00

(52) U.S. Cl. 424/543; 424/548; 71/21; 71/19

(57) ABSTRACT

A method of determining an effective method for control of soil pathogens in a soil which includes the step of measuring the pH of the soil, measuring the organic carbon content of the soil, measuring the buffering capacity of the soil, adding a nitrogen containing material and a pH reducing agent to reduce the soil pH below 5.5 when the buffering capacity of the soil is below a predetermined amount, and adding a nitrogen containing material and a pH raising agent to raise the pH above 8.5 when the organic carbon content is less than 1.7% by weight.

This application is a Continuation-in-Part Application of U.S. application Ser. No. 09/624,098 filed Jul. 24, 2000.

### FIELD OF THE INVENTION

The present invention relates to the control of soilborne pathogens and to a method for determining the treatment of a soil containing soilborne pathogens.

### BACKGROUND OF THE INVENTION

The treatment of soil in agricultural systems is widely practiced and essential since the growing of plant life remains a basic requirement for feeding the world population. Over the years, many different types of soil treatments have been used and experimented with depending on the desired result. Thus, it is common and widely known to use fertilizers to provide the nutrients for plant growth.

A common problem associated with most crops is the presence of soil pathogens and weeds within the soil. These soil pathogens substantially reduce the yields of any given crop and accordingly, many different soil treatments have been developed in order to rid the soil of such pathogens.

One of the more common and widely used methods of treating soils to eliminate pathogens therein is by the use of a methyl bromide. However, methyl bromide has been recognized as an ozone depleting chemical and as such, international agreement has stated that all production in developed countries must be phased out by the year 2005.

It has been estimated that the ban on methyl bromide will have a serious effect on crop damages and yields. In particular, crops such as potatoes, tomatoes, peppers, strawberries, etc are particularly susceptible to soilborne pathogens.

While other products for treating soil have been proposed, they have not received any wide degree of acceptance. Other methods of controlling soil pathogens include crop rotation and field fallowing. However, these approaches lower the return on a given parcel of land.

### SUMMARY OF THE INVENTION

It is therefore an object of the present invention to provide a method for controlling soil pathogens.

It is a further object of the present invention to provide a method to determine an effective treatment for soil pathogens.

According to one aspect of the present invention, there is provided a method of controlling soilborne pathogens in a soil which comprises the step of adding a nitrogen containing material and a pH reducing agent to the soil, the pH reducing agent being present in an amount sufficient to reduce soil pH below 5.5.

In a further aspect of the present invention, there is provided a method of determining an effective method for control of soil pathogens in a soil comprising the steps of measuring the pH of the soil, measuring the organic carbon content of the soil, measuring the buffering capacity of the soil, adding a nitrogen containing material and a pH reducing agent to reduce soil pH below 5.5 when the buffering capacity is below 2 uL H₂SO₄ g/soil, and adding a nitrogen containing material at a pH raising agent to raise the pH above 8.5 when the organic carbon content is less than 1.7% by weight.

In a further aspect of the present invention, there is provided a method of controlling soilborne pathogens in a soil having an organic carbon content less than 1.7% by weight comprising the step of adding a nitrogen containing material and a pH raising agent to raise soil pH above 8.5.

It is being found that the most effective method of controlling soil pathogens will depend upon key properties of the soil. In particular, it is being found that soilborne pathogens can be controlled by exposing the pathogens to either ammonia or nitrous acid in a sufficient concentration, one method being selected over the other depending upon soil properties.

In particular, it is known that ammonia is effective for the control of soilborne pathogens. However, it has been found that the addition of even large amounts of a nitrogen containing compound sufficient to generate ammonia will not function when the pH of the soil is at below critical level and when the organic carbon content of the soil is greater than 1.7% on a weight basis.

Similarly, it has been found that nitrous acid (HNO₂) and/or nitrite (NO₂) is effective when the pH of the soil is reduced to a level below 5.5 and preferably below 5.

The fungus Verticillium dahliae Kleb., a wilt pathogen of many crops, is used as a model pathogen. In potato (Solanum tuberosum), Verticillium wilt causes premature senescence and the disease is aptly referred to as “early dying syndrome”. Infection of potato plants occurs when roots contact microsclerotia (MS) of V. dahliae. Microsclerotia over winter in soil and consist of clustered, melanized, thick-walled and hyaline, thin-walled hyphal cells. Verticillium wilt is difficult to manage because of the limited success of crop rotations, the slow development of resistant cultivars and the absence of chemical control options. Our studies and those of others have shown that when one controls this organism in the soil, then many other pathogenic agents, nematodes, weeds and pests are similarly controlled.

The following examples and figures examplify different aspects of the invention, wherein;

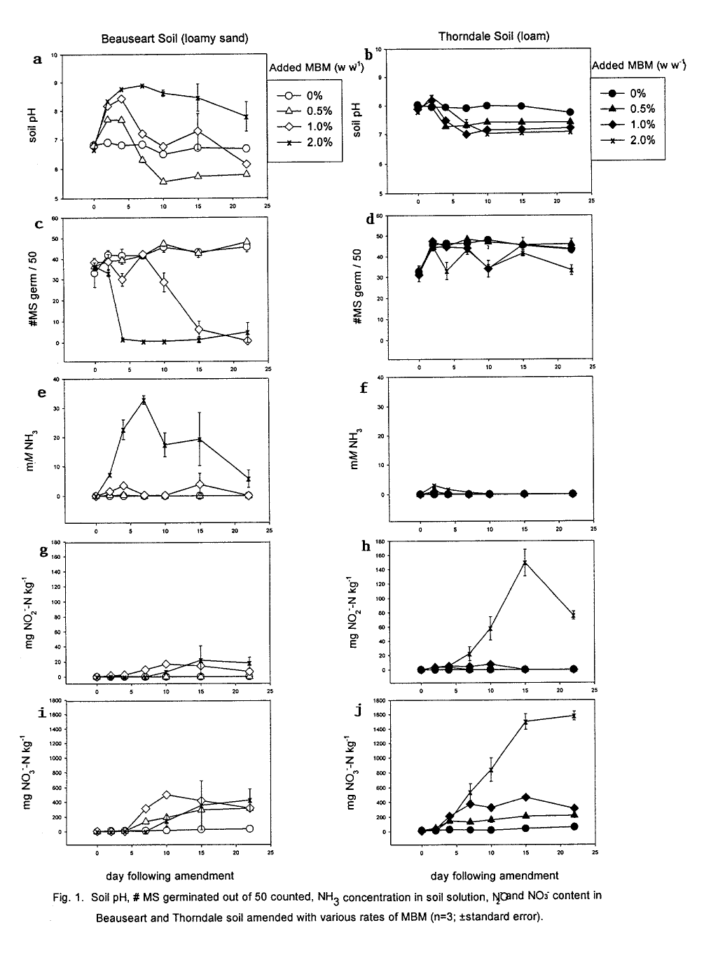

FIGS. 1a to 1j are graphs indicating soil pH and the number of microsclerotia germinated as well as NH₃ concentration in soil, NO₂⁻ and NO₃⁻ content in two different soils;

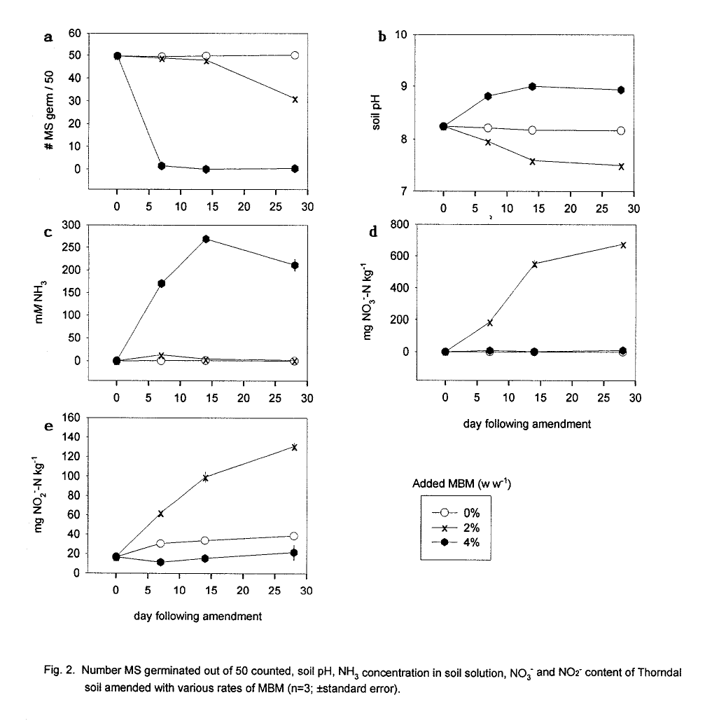

FIGS. 2a through 2e are graphs showing microsclerotia germinated in a single soil;

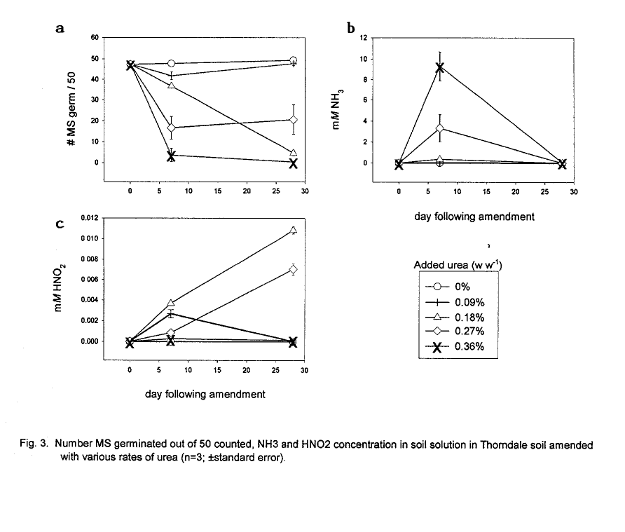

FIGS. 3a, 3b and 3c are graphs showing microsclerotia germinated in a soil amended with various amounts of urea;

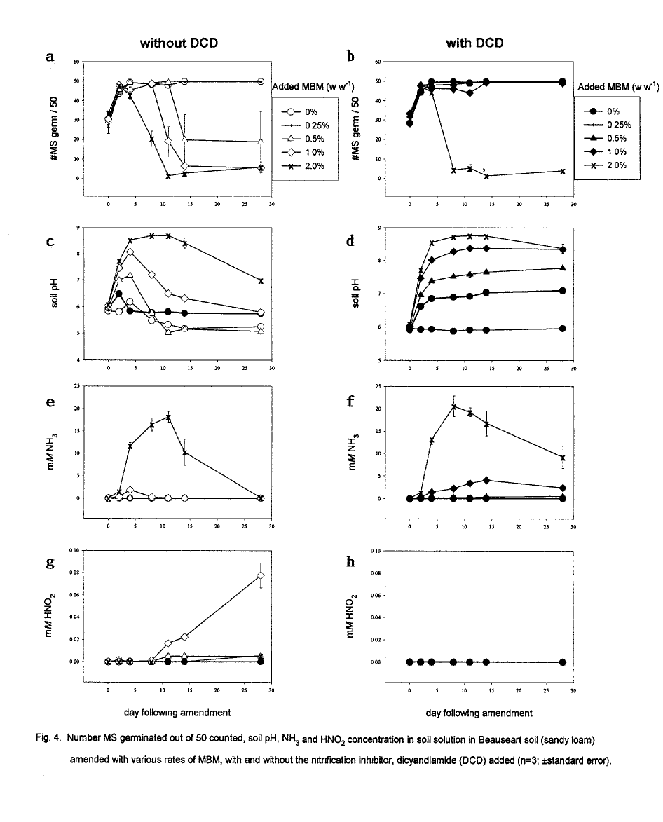

FIGS. 4a through 4h are graphs showing the effect with and without the nitrification inhibitor DCD;

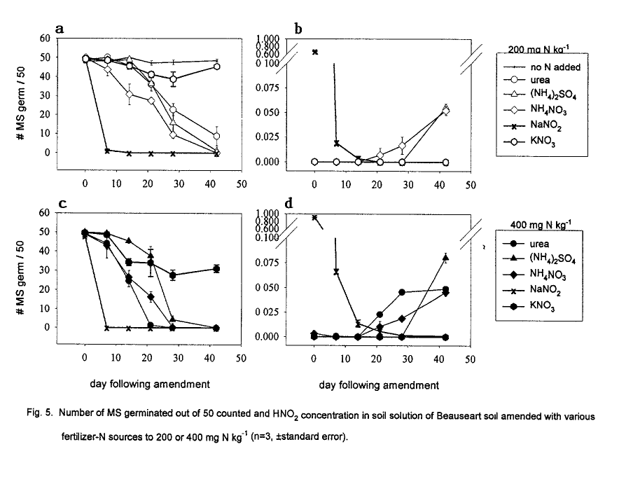

FIGS. 5a through 5d are graphs showing the number of microsclerotia germinated and HNO₂ concentration;

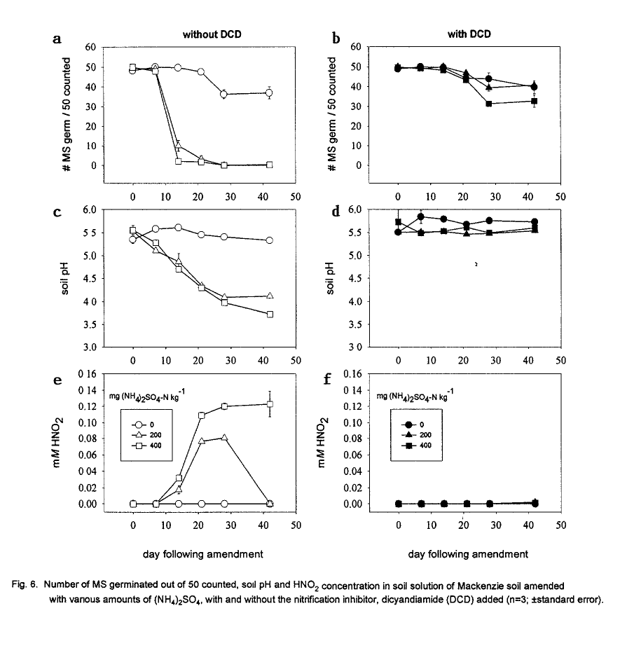

FIGS. 6a to 6f are graphs showing microsclerotia germinated, soil pH and HNO₂ concentration of a soil amended with various amounts of (NH₄)₂SO₄, with and without a nitrification inhibitor;

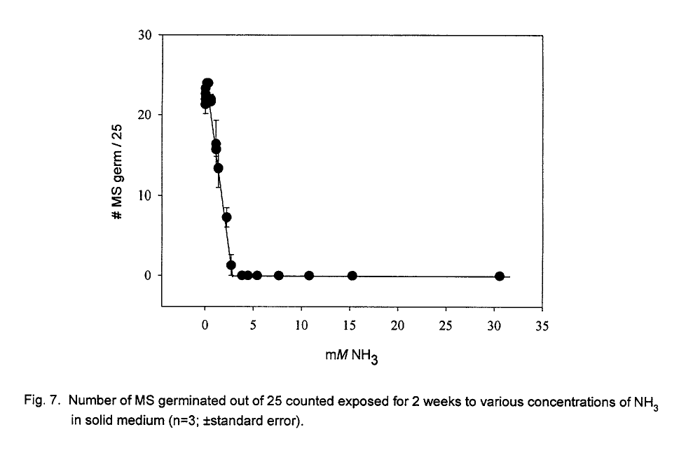

FIG. 7 is a graph illustrating the number of microsclerotia germinated after being exposed for two weeks to various concentrations of NH₃;

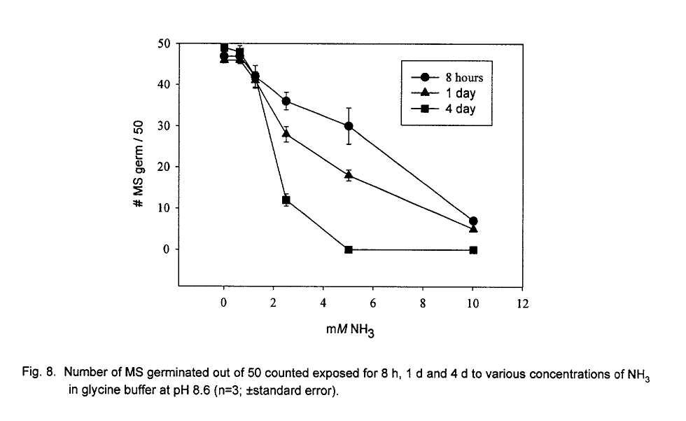

FIG. 8 is a graph illustrating the number of microsclerotia germinated for various time counts after exposure to various concentrations of NH₃;

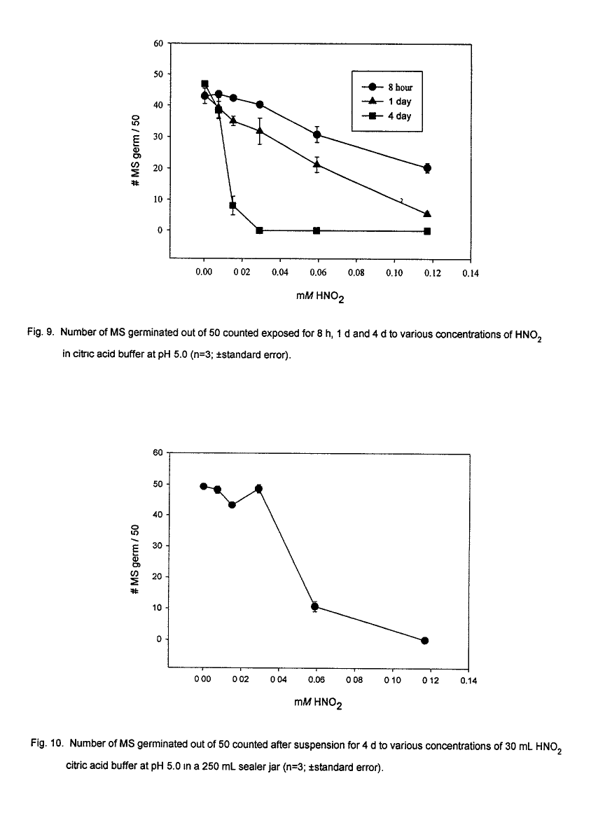

FIG. 9 is a graph illustrating the number of microsclerotia germinated at various times after being exposed to various concentrations of HNO₂ and a citric acid buffer;

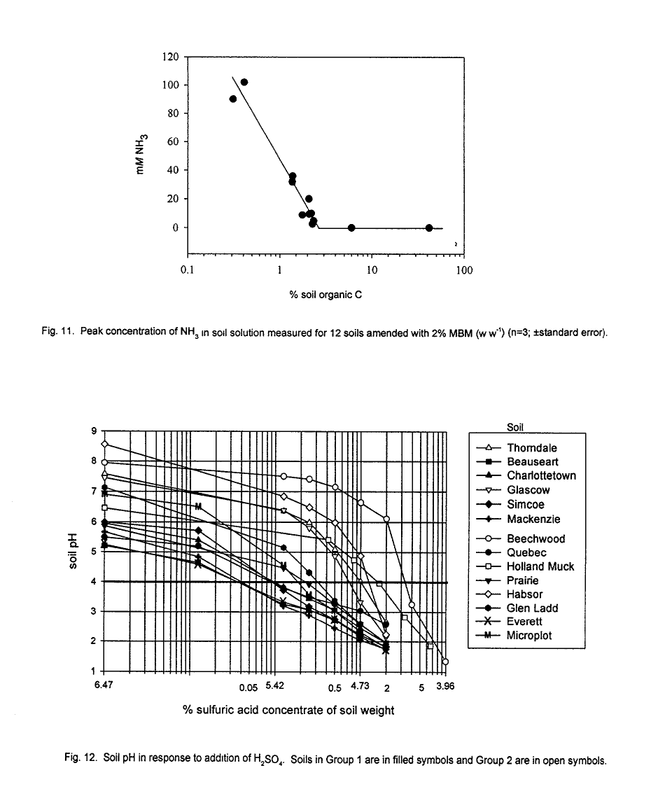

FIG. 10 is a graph illustrating the number of microsclerotia germinated after exposure to various concentrations of 30 mL HNO₂ and a citric acid buffer;

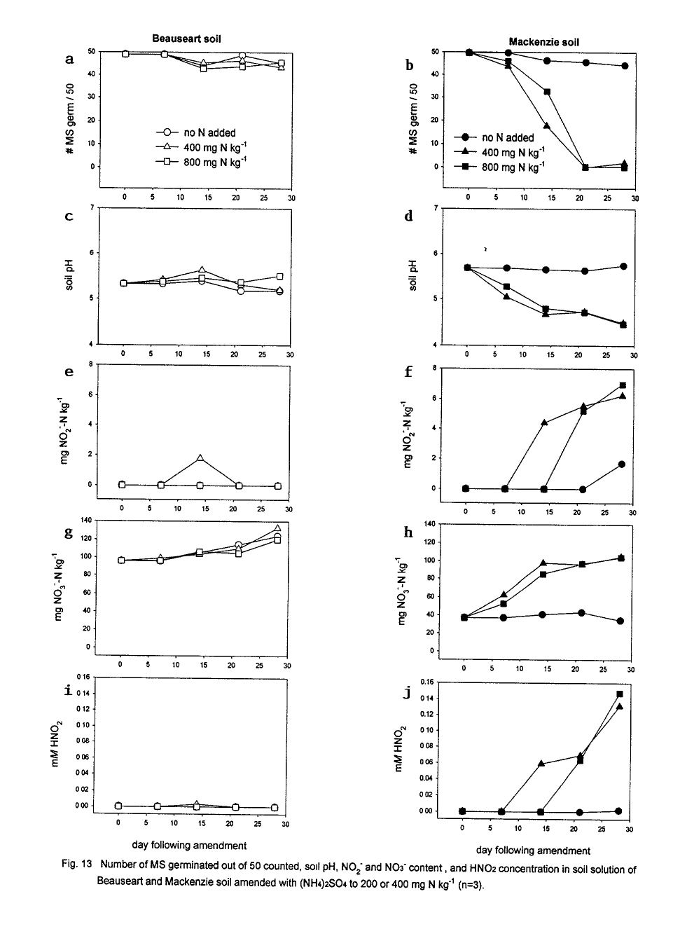

FIG. 11 is a graph illustrating the peak concentration of NH₃ for a soil amended with 2% MBM;

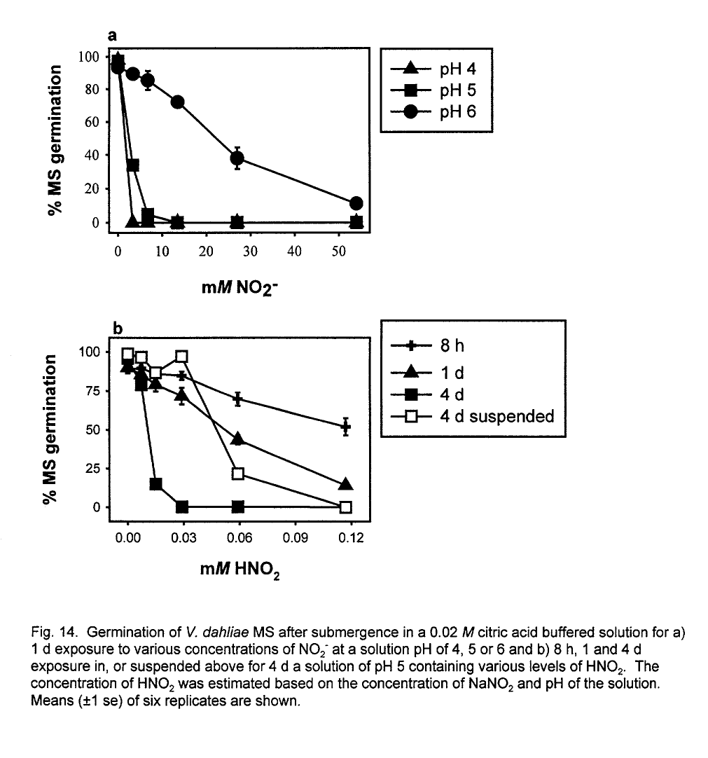

FIG. 12 is a graph illustrating soil pH in response to H₂SO₄;

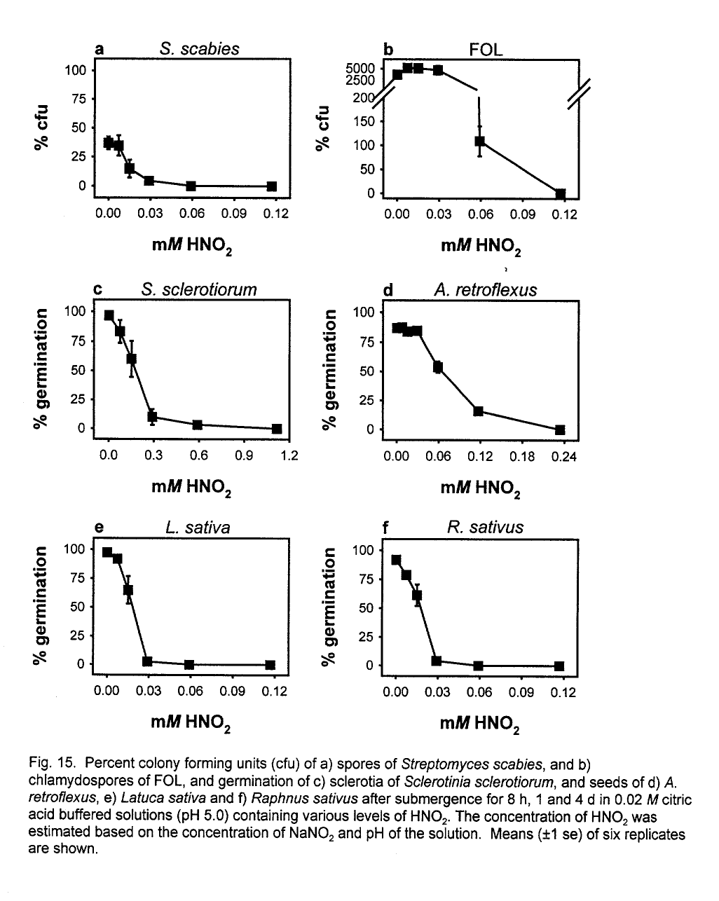

FIGS. 13a through 13j are graphs illustrating the number of microsclerotia germinated, soil pH, NO₂⁻ and NO₃⁻ content and HNO₂ concentration in a soil solution and two different soils;

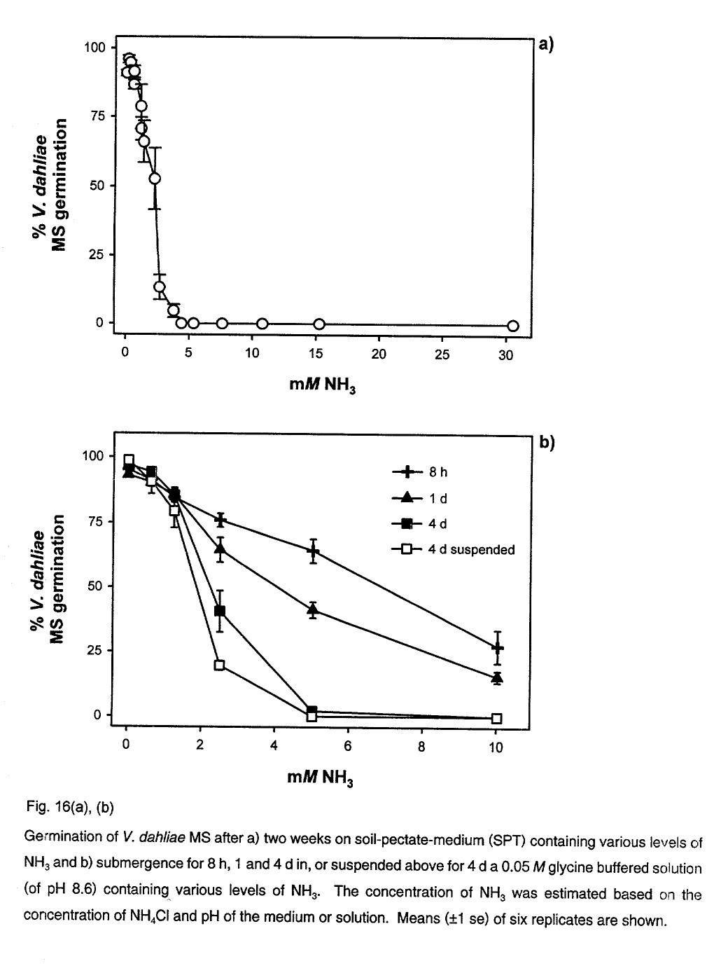

FIGS. 14a and 14b show the germination of microsclerotia after submergence in a citric acid buffered solution at differing pHs;

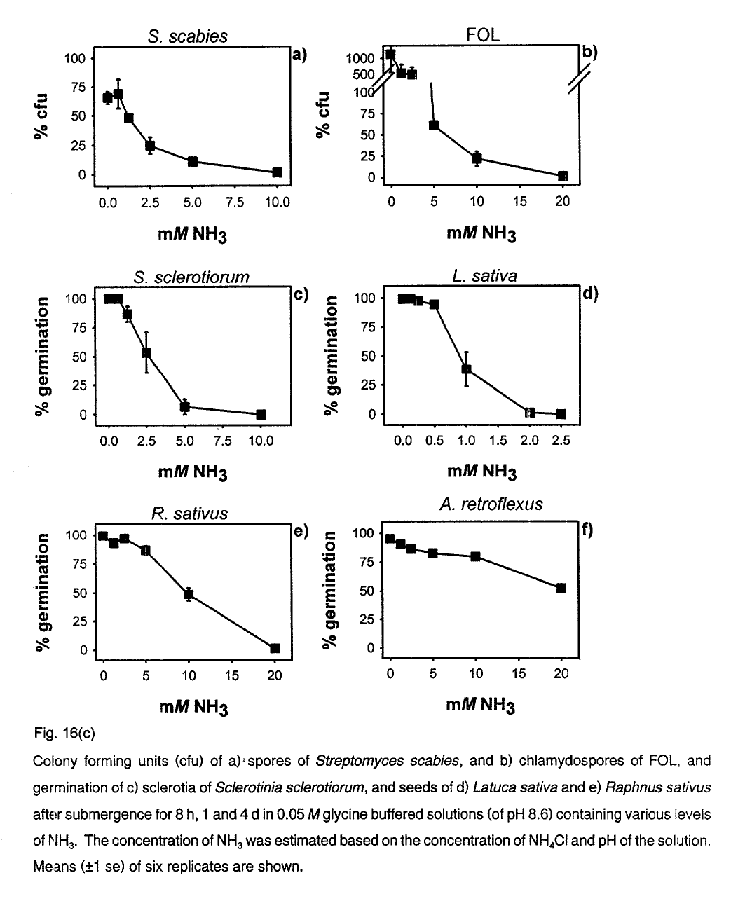

FIGS. 15a to 15f show percent colony forming units of different types of spores after submergence in citric acid buffered solution containing various levels of HNO₂;

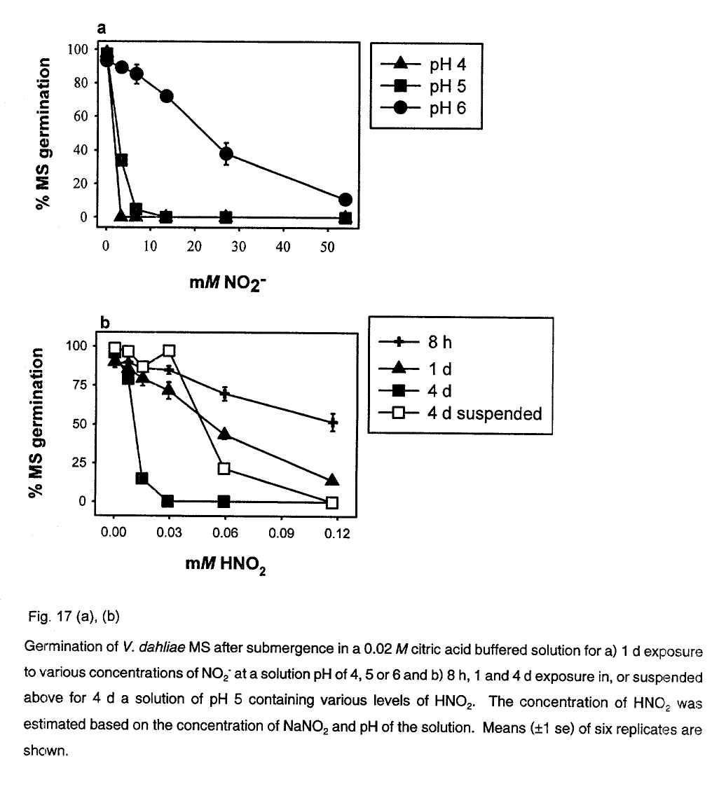

FIGS. 16a, 16b, 16c are graphs showing the germination of microsclerotia subjected to various levels of ammonium; and

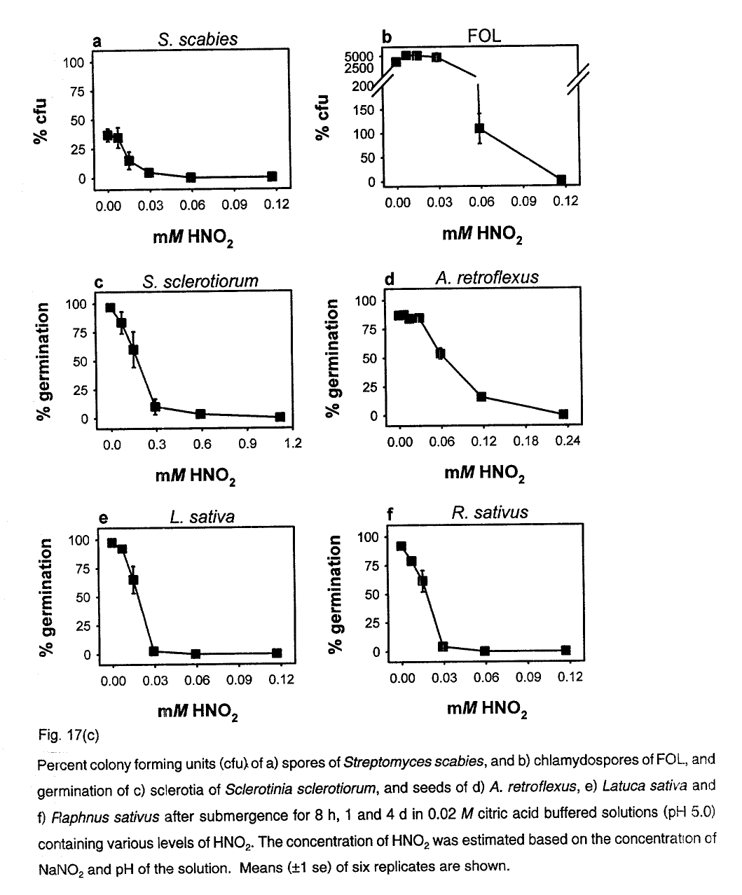

FIGS. 17a, 17b and 17c are graphs showing the germination of microsclerotia after submergence in a citric acid buffered solution.

The viability of V. dahliae microsclerotia retrieved from soil was determined using the following experimental model system. Desired levels of nitrogenous product comprising meat and bone meal (MBM) were mixed with soil and 20 g of the mixture added to 50 mL test tubes. Microsclerotia suspended in crushed silica sand was added to a nylon pouch which was then buried in the soil or suspended in the head space of each tube. Water was added to each tube to bring soil moisture to 0.33 bar tension. The tubes were then loosely capped, and placed in the dark at 24° C. On each date of analysis, the nylon pouches were retrieved and an Anderson air sampler used to impact the pouch contents onto a medium (soil-pectate-tergitol) selective for growth of V. dahliae. Viability of microsclerotia were determined two weeks following plating as the number of microsclerotia germinated from a total of 50 examined. Resulting death of microsclerotia using this bioassay has been shown to correlate to the reduction of wilt disease incidence in greenhouse and field planted potatoes.

The levels of NH₃, NO₂⁻ and NO₃⁻ in soil were determined at each sample date. Soil (8 g) was mixed with cold distilled water (40 mL) in sealed plastic bags, the slurry mechanically disrupted with a Stomacher laboratory blender, and shaken at 5° C. for one hour. The slurry was once again mechanically disrupted and its pH determined. The slurry was centrifuged and supernatant analyzed for total (NH₃+NH₄⁺), (NHO₂+NO₂⁻) and NO₃⁻ using an ion chromatograph. Ammonia and HNO₂ were calculated as the fraction of total (NH₃+NH₄⁺) or (NO₂⁻+HNO₂) respectively in solution using the Henderson-Hasselbalch equation knowing soil pH and incubation temperature.

Ammonia in excess of 65 mg N kg⁻¹ soil (20 mM NH₃) coincided with a rapid loss in the viability of microsclerotia (FIG. 1). In two experiments MBM or soya meal (SM) were added to various concentrations (0, 0.25, 0.5, 1, and 2% weight/weight) to soils from two locations namely, Beauseart and Thorndale. Quite high levels of ammonia accumulated in the Beauseart soil amended to 2%, but none was detected in the Thorndale soil. The viability of microsclerotia remained above 60% in Thorndale soil compared to less than 10% in Beauseart soil amended to 2% (weight/weight). When 1% MBM or SM was added to Beauseart soil a gradual decline in microsclerotia viability to 0% was seen four weeks after amendment. The decline in microsclerotia viability coincided with decreasing soil pH from 8 to 6 and NO₃⁻ accumulation in soil. Ammonia accumulation was negligible in Beauseart soil amended to 1% MBM (weight/weight), suggesting it was not involved in the death of microsclerotia at this rate. The results suggest the microsclerotia were killed by acute NH₃ toxicity in Beauseart soil amended to 2% (weight/weight) with MBM or SM and by quite a different mechanism when amended to 1%.

The Thorndale soil amended to 2% MBM or SM failed to accumulate sufficient NH₃ to kill microsclerotia. This provided the opportunity to confirm NH₃ as responsible for killing of microsclerotia by inducing high levels of NH₃ in the Thorndale soil by determining the survival of microsclerotia. This approach consisted of adding high rates of MBM to the Thorndale soil. Thus MBM was applied at the rates of 0,2 and 4% (weight/weight). The 2% amendment resulted in negligible NH₃ accumulation and survival of microsclerotia greater than 50% by the end of the study (FIG. 2). In contrast at 4% MBM, NH₃ accumulated to above 150 mM one week following amendment and continued to the end of the study. This corresponded to complete death of microsclerotia.

Accumulation of greater than 10 mM NH₃ was consistently accompanied with a rapid decline in the viability of microsclerotia in soil. Based on this observation the toxicity of NH₃ to microsclerotia in solution and atmosphere was tested and compared to levels required in soil to kill microsclerotia in soil.

Germination of microsclerotia was prevented by NH₃ concentrations larger than 3 mM in agar medium (FIG. 7). NH₃ was generated in SPT medium by addition of various concentrations of NH₄Cl to the medium and varying the pH of the medium (7, 7.6, 8 and 8.5). Immediately following cooling and hardening of the medium, 25 microsclerotia per Petri dish were transferred individually with a needle and germination recorded two weeks later. Microsclerotia that failed to germinate were transferred to regular SPT (containing no NH₃) and still did not germinate.

Various concentrations of NH₃ were generated in glycine or tricine buffer solution at pH 8.6 with NH₄Cl or (NH₄)₂SO₄ added. A 15 mL test tube was filled with appropriate buffer and NH₄⁺ solution, microsclerotia added, the tube capped, then placed in the dark at 24° C. and the tubes inverted daily to suspend the microsclerotia in solution. Microsclerotia survival was determined by emptying contents of the tube into a Buchner funnel, the microsclerotia being retained on Whatman #42 filter paper, rinsed with distilled water, transferred to SPT medium by placing the filter paper in contact with the agar medium and removed leaving the microsclerotia adhering to the medium.

Microsclerotia germination decreased with concentration and duration of exposure to NH₃ in glycine buffer (FIG. 8). An exposure of four days to greater than 5 mM NH₃ prevented germination of microsclerotia. Germination of microsclerotia to various concentrations of NH₃ was not affected by buffer (glycine or tricine), NH₄⁺ source (NH₄Cl or (NH₄)₂SO₄) or NaCl concentrations equivalent to the N sources added.

In previous experiments, NH₃ was found to accumulate in the Thorndale but not Bearseart soil amended to 2% MBM (weight/weight). Therefore a series of experiments were conducted to determine the soil properties preventing NH₃ accumulation and microsclerotia death in soil.

Several studies have reported that soil organic matter or clay can absorb NH₃ to exchangeable negatively charged sites. Thus various amounts of NH₄0H were added to either soil (varying in levels of NH₃ eq. to 0 to 4% MBM weight/weight), soil brought to 0.333 bar, incubated for four days with subsequent extraction for estimation of NH₃. Addition of about 1800 mg NH₄OH—N kg⁻¹ (eq. to 2 MBM weight/weight) to the Thorndale or Beauseart soil was sufficient to induce levels of NH₃ sufficient to kill microsclerotia (data not shown). Therefore, it seems retention of NH₃ in amended Thorndale soil cannot explain the occurrence of insufficient NH₃ levels to kill microsclerotia.

Rapid nitrification was observed in the Thorndale soil amended to 2% MBM To test if this rapid nitrification converted NH₃ to NO₂⁻ and NO₃⁻, thus preventing NH₃ accumulation, the nitrification inhibitor dicyandiamide (DCD) was added with MBM to the Thorndale soil. Addition of inhibitor prevented the accumulation of NO₂⁻ or NO₃⁻ and the reduction in soil pH following amendment to 2% MBM However, NH₃ levels in soil were not sufficient to kill microsclerotia, thus nitrification alone cannot be attributed to prevention of NH₃ toxicity in the Thorndale soil (data not shown).

To determine the factor(s) that control NH₃ accumulation in soil, 2% MBM was added to twelve soils with a range of soil properties including texture, organic carbon, cation exchange capacity, NH₃ and acid buffering capacity. The Beauseart and Thorndale soils studied previously were included in the twelve soils. Selected soil properties including NH₃, NH₄⁺, NO₂⁻, NO₃⁻, pH, soil C:N ratio, electrical conductivity, total bacteria, total fungi, ammonifying bacteria, ammonifying fungi, proteolytic bacteria and soil respiration which may influence the accumulation of NH₃ in soil were measured over time. Microsclerotia were killed within one week of addition in four of the twelve soils amended. An accumulation of NH₃ (greater than 65 mg N kg⁻ or 20 mM) was found in each of these soils one week following amendment. Organic carbon content of soil was the only soil property highly related to NH₃ accumulation (r=0.92) with each of the four soils containing less than 1.7% organic carbon (weight/weight) (FIG. 11).

Ammonia failed to accumulate to toxic levels in soils with an organic carbon content larger than 1.7% amended to 2% MBM. To confirm the role of organic carbon in soils controlling the level of NH₃ accumulation following amendment, a recalcitrant source of carbon (Holland Marsh Muck soil) was added to Beauseart and Habsor (sand) soils and amended with MBM. Amendment of the Beauseart soil to 2% MBM resulted in death of microsclerotia by day 9 with associated high levels of NH₃ in soil. In contrast addition of Holland Marsh soil to 2 and 4% (weight/weight) together with MBM to 2% in Beausaert soil resulted in survival of microsclerotia with negligible levels of NH₃ present in soil (data not shown). Addition of Holland Marsh soil to 5% (weight/weight) of the Habsor sand amended to 2% MBM (weight/weight) resulted in greater than 80% survival of microsclerotia with an insufficient amount of NH₃ in soil (less than 15 mg NH₃—N kg⁻¹ or 7 mM NH₃) required to kill microsclerotia (data not shown).

The lack of NH₃ accumulation in the Thorndale soil amended with MBM or SM was attributed to soil pH remaining below 8.5, being insufficient to convert NH₄⁺ to NH₃. Calcium oxide (CaO) stabilized sewage sludge (pH 13) was added to soil to raise soil pH and induce NH₃ toxicity. The sludge raised soil pH above 8.5, during the first four days following its addition. Only when the sludge was added five days following MBM amendment were microsclerotia killed (Table 1). The NH₄⁺ released during decomposition and mineralization of MBM at day 5 was converted to NH₃ by the liming effect of the CaO stabilized sludge, thus NH₃ toxicity induced.

TABLE 1
Number of microsclerotia germinated out of 50 counted with 0 or
2% added and 0 or 4% CaO stabilized municipal sewage sludge added
on day 0 or day 5 after M.M. addition.

| % | % | Day of | MS germination |
|---|---|---|---|
| MBM | Sludge | Sludge | (N = 3 standard |
| (weight/weight) | (weight/weight) | Addition | error of the mean) |
| 0 | 0 | — | 50 (0.33) |
| 0 | 4 | 0 | 48 (1.15) |
| 2 | 0 | — | 45 (0) |
| 2 | 4 | 0 | 44 (1.53) |
| 0 | 0 | — | 46 (0.33) |
| 0 | 4 | 5 | 42 (2.19) |
| 2 | 0 | — | 47 (1.20) |
| 2 | 4 | 5 | 0 (0) |

Base generating agents such as calcium hydroxide, calcium oxide, sodium hydroxide, ammonium hydroxide, and potassium hydroxide can be added to various soils to increase soil pH and encourage the generation of NH₃ from N amendments. By doing so the required rate of N amendment to disinfect soil of soilborne pathogens will be reduced to economical and environmentally suitable levels. Further, the amount of the base agents required to bring soil pH to desired levels to induce generation of NH₃ can be determined. Soil properties such as organic matter content and initial soil pH are being used to predict the amount of base agent and N amendment required to disinfest soil of plant pathogens and pests.

The germination of V. dahliae MS having been exposed to varying concentrations of NH₃ was determined. V. dahliae MS were exposed to NH₃ in solid agar medium, in buffered solutions, and in the atmosphere above buffered solutions containing NH₃. All toxicity studies were done in triplicate and repeated once.

Agar Medium:

Various amountes of NH₄Cl salt (to 0, 25, 50, 100 and 200 mM) was added to soil peclate medium (SPT) and the pH of the medium adjusted to 7.0, 7.6, 8.0 or 8.5 by addition of 5 M NaOH. For each level of NH₄Cl and pH, three replicate dishes were poured. The agar was allowed to solidify for 2 hours and immediately thereafter 25 V. dahliae MS were individually transferred to each dish using a sterile hypodermic needle. The dishes were wrapped with Parafilm (American National Can, Neenah Wis.) to limit loss of NH₃ by volatilization, and incubated for two weeks at 24° C. in the dark. The viability of V. dahliae MS was determined as percent of total MS forming colonies. The concentration of NH₃ in medium was estimated as described below and ranged from 0 to 31 mM. The pH of medium after hardening and two weeks after incubation was tested using pH test strips (ColorpHast pH 5-10; EM Science, Gibbstown N.J.) and was within 0.5 units (limit of resolution of the test strips) of that set at the start. The results are shown in FIG. 16(a).

Buffered Solutions:

Varying amounts of a 2.70 M NH₄Cl stock solution was added (from 0 to 1.0 mL) to 40 mL of 0.05 Mglycine solution adjusted to pH 8.6 with NaOH (Perrin & Dempsey 1974). The final volume was brought to 50 mL with the same glycine solution. Solutions were then sterilized by filtration through a 0.22 um pore size filter and 15 mL placed into three replicate sterile screw cap tubes (total capacity 15.5 mL) About 200 V. dahliae MS were immediately added to each tube and the tube closed and placed at 24° C. and in the dark. Tubes were inverted every 12 hours to mix and suspend V. dahliae MS in the solution. A maximum duration of exposure of four days was chosen because V. dahliae MS germinated after five days in solutions of 0 to 0.65 mM NH₃. At 8 hours, 1 and 4 days, the contents of each tube was passed onto a Buchner funnel containing sterile filter paper (Whatman #42). The filter paper retained the V. dahliae MS and was rinsed with sterile water and placed onto the surface of SPT medium such that V. dahliae MS contacted the agar surface. The paper was then removed, leaving V. dahliae MS adhering to the surface of the medium. The viability of V. dahliae MS was determined as the percentage of MS forming colonies out of 50 counted per replicate dish. The concentration of NH₃ in solutions was estimated as described below. The effect of type of buffer solution or NH₄⁺ salt on survival of V. dahliae MS was tested by adding (NH₄)₂SO₄ to glycine buffer solutions instead of NH₄Cl, NaCl instead of NH₄Cl to glycine buffered solutions, and NH₄Cl added to tricine buffered solutions. The solution concentration of NH₄⁺+NH₃ in tubes after 4 days was within 5% of that measured at the start of the assay. Solution pH after 4 days did not vary from the set pH at the start of the assay by more than 0.1 units. The results are shown in FIG. 16(b).

Ammonia Gas:

Fifty mL of a prepared solutions of NH₄Cl in glycine solution described previously were added to sealer jars (each 250 mL). A mesh bag containing V. dahliae MS was then suspended in the atmosphere of the jar using a paper clip attached to a septum fitted in the lid of the jar. The jar was sealed, and placed at 24° C. in the dark for 4 days. The mesh bags were retrieved and the viability of V. dahliae MS determined.

The survival of the other test organisms exposed to NH₃ was determined in glycine buffered solutions prepared as described previously. Sclerotia of S. sclerotiorum (15 per treatment), seeds of A. retroflexus, L. sativa and R. sativus (50 to 100 per treatment) were added to each tube containing a test solution. For S. scabies and FOL a one mL suspension of spores and chlamydospores respectively were added to each tube containing 14 mL of concentrated NH₄Cl/glycine solution. The propagule density in the added suspension was prepared in distilled water and adjusted to give about 50 colony forming units (cfu) per 0.1 mL of test solution at the start of the experiment. Test solutions containing S. scabies and FOL were placed on YME and PDA medium respectively. Three replicate platings were maded for each test solution of S. scabies and FOL and the cfu count were averaged. The viability of sclerotia of S. sclerotiorum was determined as colony formation (non-carpogenic germination) on PDA medium and that of seeds on water agar (WA) medium (1.5% agar) in Petri dishes. Sclerotia and seeds were separated from test solutions in a similar manner as that for V. dahliae using a Buchner funnel and sterile filter paper. Sclerotia were cut in two, and a total of 5 placed (cut surface down) onto a dish containing medium. Seeds were transferred to WA medium directly from the filter paper as done for V. dahliae. Dishes were immediately wrapped using stretchable sealer tape. All dishes containing propagules of the test organisms were placed at 24° C. in the dark. Seed survival was based on the development of a 2 mm radicle. Seeds of R. sativus that failed to germinate after exposure to NH₃, were checked for viability by staining with tetrazolium salt (2:3:5-tripenyl-tetrazolium chloride; BDH, Poole UK) according the procedure outlined by Moore (1973). The viability of all organisms tested was expressed as a percentage of that determined for the 0 mM test solution at the start of the experiment. For each test organism, this experiment was done in triplicate and repeated once. The results are shown in FIG. 16(c).

The germination of V. dahliae MS was determined using a setup similar to that described above for exposure of V. dahliae MS to NH₃ in buffered solution. Exceptions being various amounts of 0.270 M NaNO₂ stock solution were added (from 0 to 2.0 mL) to 40 mL of 0.02 M citric acid solution (set to pH 5.0 with NaOH (Perrin and Dempsey 1974)) and brought to 50 mL volume with the same citric acid solution. All experiments were done in triiplicate and repeated. Solutions were filter sterilized and 15 mL placed into sterile screw cap tubes (total capacity 15.5 mL). Immediately, V. dahliae MS were added to each tube, the tube closed, placed at 24° C. and in the dark and inverted twice daily. At 8 hours, 1 and 4 days, V. dahliae MS viability was determined. The concentration of HNO₂ in solutions was estimated as described below. The effect of NO₂⁻ on survival of V. dahliae MS was tested by adding various amounts of NaNO₂ (0 to 53 mM NO₂⁻) to citric acid buffer and pH set to 4.0, 5.0 and 6.0. The effect of type of NO₂⁻ salt on survival of V. dahliae MS was tested by adding KNO₂ or NaCl instead of NaNO₂ to citric acid buffer (set to pH 5.0). The concentration of NO₂⁻+HNO₂ and pH of solutions after 4 days was within 5% and 0.1 units of the solution at day 0, respectively. The results are shown in FIG. 17(a).

Exposure of V. dahliae MS to HNO₂ gas was done using a similar procedure described above. To sealer jars (250 mL), 50 mL of a prepared solution of NaNO₂ in citric acid solution was added. A V. dahliae MS bag was suspended in the atmosphere of the jar. The jar was sealed, and placed at 24° C. in the dark for 4 days. Thereafter the bags were retrieved and the viability of V. dahliae MS determined. This experiment was repeated and done in triplicate. The results are shown in FIG. 17(b).

Determination of the survival of the other test organisms exposed to HNO₂ was done using citric acid buffered solutions as described above and detailed by Tenuta and Lazarovits (2000a). Sclerotia of S. sclerotiorum, chlamydospores of FOL, spores of S. scabies, seeds of A. retroflexus, L. sativa and R. sativus were added to each tube containing 15 mL of HNO₂, in citric acid solution at pH 5.0. Tubes containing seeds were opened for 15 s on day 2, as a precaution to prevent generation of anaerobic conditions from respiration of seeds. The solution assay was done twice and in triplicate for each organism tested.

Seeds of R. sativus that failed to germinate after exposure to HNO₂, were checked for their viability by staining with tetrazolium salt (2:3:5-tripenyl-tetrazolium chloride; BDH, Poole UK) according the procedure outlined by Moore (1973). The germination or cfu of all organisms tested was expressed as a percentage of the germination or cfu of a control solution (0 mM HNO₂) at the start of the experiment. The results are shown in FIG. 17(c).

Various concentrations of HNO₂ were generated in citric acid buffer solution at pH 4.0, 5.0 or 6.0 with NaNO₂ added. A 15 mL test tube was filled with appropriate buffer and NaNO₂ solution, microsclerotia added, the tube capped, placed in the dark at 24° C. and tubes inverted daily to suspend microsclerotia in solution. Microsclerotia survival was determined as described previously for NH₃ in glycine buffer. Microsclerotia survival decreased with increasing concentration of NaNO₂ and decreasing pH at a 24 hour exposure. A calculated concentration of above 0.10 mM HNO₂ was required to kill all microsclerotia (data not shown). Further, the survival of microsclerotia was dependent upon the duration of exposure to HNO₂, about 0.025 mM HNO₂ was sufficient to kill all microsclerotia at a four day exposure (FIG. 9). This is within the range of critical HNO₂ concentration required in soil to kill microsclerotia.

In studies described here, microsclerotia died when suspended above soil amended with MBM, SM, and various fertilizers. Therefore, HNO₂ was suspected to also kill microsclerotia in atmosphere. Various amounts of HNO₂ in atmosphere were subjected to microsclerotia by suspending microsclerotia in sealer jars containing 30 mL of citric acid HNO₂ buffer solution and incubated in the dark at room temperature for four days. Microsclerotia died when suspended in atmosphere above the 0.10 mM HNO₂ solution (FIG. 10).

This finding is important because it demonstrates that both NH₃ and HNO₂ can kill microsclerotia through exposure in soil solution or soil atmosphere. Since in soil microsclerotia may reside in both, solution and atmosphere, either compound has the potential to kill microsclerotia.

Observations from the experiments described here indicate three factors determine nitrous acid toxicity in soil. They being: a) amendment rate, b) rapid nitrification and c) poor soil acid buffering capacity. Nitrification determines nitrous acid toxicity because the intermediate NO₂ is produced under conditions of rapid nitrification and the oxidation NH₄⁺ to NO₂⁻ generates protons which acidifies soil. The ability of a soil to buffer against the acidity generated during nitrification determines the relative amounts of HNO₂ and NO₂⁻ according to soil pH. A acid buffering assay was developed in which various amounts of H₂SO₄ were added to soil, incubated for two hours, distilled water added and the slurry shaken for one hour, the slurry then being allowed to settle for one hour with subsequent pH determination. Generally, soils group into two categories as may be seen in FIG. 12. Group 1 soils have the ability to accumulate HNO₂ and thus toxicity to microsclerotia. Soils in this group require less than 2 uL H₂SO₄ g/soil to lower soil pH to 5. Those in Group 2 are soils in which greater than 6 uL H₂SO₄ g/soil weight is required to lower soil pH to 5. HNO₂ acid accumulation and toxicity to microsclerotia has not been demonstrated in soils belonging to this group. Soils in this group contain CaCO₃ this being the source of their buffering ability.

An example of the importance of nitrification rate in producing HNO₂ is evident in a study in which 400 or 800 mg N kg⁻¹ as (NH₄)₂SO₄ was added to Beauseart and Mackenzie soils. The Beauseart soil was air-dried and stored for 1.5 years prior to initiation of the experiment. The Mackenzie soil was recently collected and stored at 4° C. and at field moisture content. Recently collected Beauseart soil was shown previously to generate HNO₂ in response to (NH₄)₂SO₄ addition (FIG. 5). However, the air-dried Beauseart soil failed to accumulate HNO₂ and kill microsclerotia (FIG. 13). In comparison, the Mackenzie soil has rapid nitrification, associated reduction in soil pH, accumulation of HNO₂, and death of microsclerotia. The population of autotrophic nitrifying bacteria at the start of the experiment was higher in the Mackenzie soil (1.1×10⁵ g⁻¹ soil) compared to the Beauseart soil (5.8×10³ g⁻¹ soil) likely accounting for differences in nitrification rate between soils.

Many acid generating agents such as FeSO₄, AISO₄, S^{o}, SO₂, H₂SO₄, ascorbic, sorbic, citric and acetic acids can be added to various soils to lower soil pH and encourage the generation of HNO₂ from N amendments. By doing so the required rate of N amendment to disinfest soil of soilborne pathogens is reduced to economical and environmentally suitable levels. The amount of the acid agents required to bring soil pH to desired levels to induce generation of HNO₂ can be determined. Soil properties such as CaCO₃, sand content and initial soil pH are used to predict the amount of acid agent and N amendment required to disinfest soil of plant pathogens and pests.

It will be understood that the above described embodiment is for purposes of illustration only and changes or modifications may be made thereto without departing from the spirit and scope of the invention.

I claim:

1. A method of determining an effective method for control of soil pathogens in a soil comprising the steps of:
measuring the pH of a soil;
measuring the organic carbon content of said soil;
measuring the buffering capacity of said soil;
adding a nitrogen containing material and a pH reducing agent to reduce soil pH below 5.5 when said buffering capacity is below 2 uL H₂SO₄ g/soil; and
adding a nitrogen containing material and a pH raising agent to raise said pH above 8.5 when said organic carbon content is less than 1.7% by weight.

2. A method of controlling soilborne pathogens in a soil having an organic carbon content less than 1.7% by weight, comprising the step of adding a nitrogen containing material and a pH raising agent to raise soil pH above 8.5.

3. The method of claim 17 wherein said nitrogen containing material is selected from a group consisting of animal manures, sewage sludge, animal by-products, chitinaceous materials, oil-seed materials, urea, NH₄x and xNO₂ compounds.

4. The method of claim 17 wherein said nitrogen containing material is a meat and bone material.
